(19) United States
(12) Patent Application Publication
(10) Pub. No.: US 2007/0062811 A1
(43) Pub. Date: Mar. 22, 2007

(54) Bioelectrochemical sensor strip capable of taking trace samples

(75) Inventors: Paul Yang, Chung Ho City (TW); Ya-Hsin Chuang, Taipei City (TW); Shih-Chieh Hsiao, Taipei City (TW); Hsin-Yi Lin, Taitung City (TW)

Correspondence Address: BACON & THOMAS, PLLC, 625 SLATERS LANE, FOURTH FLOOR, ALEXANDRIA, VA, 22314, US

(73) Assignee: HEALTH & LIFE CO., LTD, CHUNG HO CITY (TW)

(21) Appl. No.: 11/230,479

(22) Filed: Sep. 21, 2005

(51) Int. Cl. G01N 33/487

(52) U.S. Cl. 204/403.01

(57) ABSTRACT

A bioelectrochemical sensor strip capable of taking trace samples includes an insulating substrate, a reacting area protruded from the edge of the insulating substrate and the periphery of the reacting area is open, and a working electrode and a reference electrode of different electrodes are disposed between the insulating substrates, and the electrodes are extended to one end of the reacting area and covered by a reacting enzyme. During a test, an end of the electrode having no reacting enzyme is inserted into a testing instrument, and the reacting area is open and the opening has no limit on the position, so that the test sample (such as blood) can be absorbed from the reacting area into the reacting area in any direction, and the reacting enzyme will be dissolved into the test sample quickly. The electrodes use an oxidizing matter or a reducing matter to have a dedicated bioelectrochemical reaction with the test sample to produce an electron transfer. The concentration of the test sample can be detected by the testing instrument. The hydrophilic reacting layer of the electrode allows the test sample to be absorbed into the reacting area easily. Since the reacting area is in a protruded form, and the opening of the reacting area has no limits on the position, therefore the test sample can enter the reacting area easily.

### BACKGROUND OF THE INVENTION

1. Field of the Invention

The present invention relates to a bioelectrochemical sensor strip, and more particularly to a sensor strip that uses an oxidizing matter or a reducing matter to have a dedicated electrochemical reaction with a test sample to produce an electron transfer. The strip and the testing instrument detect the concentration of the test sample, and the periphery of the reacting area is fully open, so that the test sample can enter into the reacting area easily without a limitation of directions.

2. Description of the Related Art

Since the concept of household nursing becomes increasingly popular, and there are various different kinds of sensor products for a fast, cheap, non-professional operation, and these sensor products include a blood sugar meter, an electronic ear thermometer, and an electronic sphygmomanometer, and the disposable blood sugar strip used for a blood sugar meter adopts the principle of a bioelectrochemical sensor to detect the blood sugar concentration of a test sample.

At present, there are many blood sugar strip products in the market and they are mainly divided into a dropping type strip and a quantitative type (capillarity type) strip according to the way of applying the blood sugar strip to a test sample. As to the design, the dropping type strip usually uses a plastic mesh to cover the reacting area for preventing contaminations, but the required quantity of the test sample is generally larger than 3 μl. The old people or senior diabetics have to collect a blood sample by a needle and agglomerate a drop of blood sufficiently to be dropped into the reacting area. This process is very inconvenient and usually causes errors and thus wasting the strips.

The required quantity of blood for the strip of a test sample required by the quantitative method (capillarity method) strip is less than 3 μl, and only a little of the test sample is needed to be contacted with the absorbing opening, and the test sample will be absorbed automatically into the reacting area. However, the design of the strip of the current siphon strip is a very small strip and thus it is very inconvenient to use the strip and very easy to contaminate the absorbing opening. Furthermore, there is only one absorbing opening in a fixed position which makes the operation very inconvenient. The required blood quantity of this strip is less than 3 μl, and the electrode surface of the reacting area is reduced to achieve the test. However, reducing the area will weaken the signal, and it is necessary to use a more precise instrument for the detection of the signals.

Referring to FIG. 1 for the U.S. Pat. No. 5,762,770, a dropping type laminated electrode strip comprises: an insulating substrate; an electrode system having two electrodes thereon; an insulating layer covered onto the electrode system and defining a reacting area and anode and cathode connector of a detector; a reacting membrane spread over in the reacting area; a plastic mesh covered onto the reacting area; and a reacting area exposed from a tape that affixes the plastic mesh.

However, the test sample of this strip is applied to the reacting area by the dropping method, and thus the dropping direction of the test sample to the reacting area is limited, and the required quantity of the test sample must be over 9 μl.

Referring to FIG. 2 for the U.S. Pat. No. 5,120,420, a capillarity type laminated electrode strip comprises an insulating substrate, an electrode system, an insulating layer and a reacting membrane disposed on the same side, and the reacting membrane forms a test sample carrier reacting area on a hydrophilic lid thereon, and the test sample carrier reacting area includes a test sample absorbing opening and a gas discharging opening, wherein the process of producing the reacting membranes comprises the steps of coating carboxymethylcellulose (CMC) solution onto the reacting area of the electrode substrate, forming a hydrophilic macromolecule CMC layer after the solution is dried; coating a glucose oxidase solution thereon, forming a GOD layer after the solution is dried, spraying hydrophilic macromolecule polyvinylpyrrolidone solution, and forming a protective layer (PVP layer) after the solution is dried, spraying an organic suspending solution including the electronic medium, and forming a reacting membrane after the solution is dried.

Firstly, the shortcomings of this capillarity type laminated electrode strip reside on that it requires four steps to produce the reacting membrane. The CMC layer is formed to improve the hydrophobic property of the electrode surface of the reacting area; the GOD layer, hydrophilic protective membrane PVP layer, and electronic medium layer are formed, and each layer has to be dried and thus making the process very complicated. Secondly, the test sample is introduced into the reacting area automatically after the test sample contacts the tip of the electrode strip, but there is only one position for inserting the test sample. Thirdly, the strip is too small and inconvenient to access, and thus such arrangement contaminates the absorbing opening of the test sample easily.

Referring to FIG. 3 for the R.O.C Pat. No. 573,735, a blood sugar test strip comprises:

a first substrate;

a first electrode, disposed on the first substrate, and having an electrode contact point at an end and an enzyme reacting area at another end;

an insulating layer, covered onto the first substrate, and the electrode enzyme acting area includes an opening;

an enzyme membrane, coated onto the electrode enzyme reacting area at the opening of the insulating layer; and

a second substrate, coved on the uppermost layer of the blood sugar test strip, and having a second electrode at the bottom, and one end of the second electrode is the electrode contact point, and the other end of the electrode is the electrode enzyme reacting area; wherein:

the insulating layer of the blood sugar test strip forms a crevice for performing the siphon effect, and an opening is formed proximate to the crevice and used as a vent opening for the siphon effect.

Although this structure can solve the foregoing problems, the opening has a specific direction, and the area is relatively small, and the direction and range of dropping the test sample are limited.

Furthermore, it is necessary to form an opening proximate to the crevice as the vent opening of the siphon effect, and thus making its use and manufacture more difficult.

Referring to FIG. 4 for the U.S. Pat. No. 6,258,229 B1, this patent has disclosed a capillarity type electrode strip that requires less than 1 μl of the test sample and comprises an insulating substrate, an electrode system having three electrodes on the same side of the substrate, a U-shape channel formed in the middle insulating layer, and an upper cover layer having a hole. However, its shortcoming resides on that the test sample only has one opening at the tip of the strip.

Referring to FIG. 5 for the R.O.C. Pat. No. 570192, a thick membrane electrode biosensor strip comprises a conductive membrane coated onto a substrate, and the conductive membrane includes a conductive silver paste and a conductive carbon paste, and the insulating layer is covered onto the substrate and the conductive membrane and a part of the conductive member is exposed, a plurality of reacting areas covered onto the exposed conductive membrane of the insulating layer, a plurality of reacting membrane layers covered onto the reacting area, a pad disposed on the reacting membrane layer, and the reacting area covered onto the insulating layer, so that a gap is maintained at the top of the reacting membrane layer for absorbing the sample, and the protective membrane is used for protecting the reacting membrane layer and covered onto the pad, and the protective membrane includes a vent opening for ventilation, and an absorbing opening pattern is disposed around the vent opening for identifying the absorbing opening.

However, its shortcoming resides on that the reacting area is located at the tip, and thus the test sample cannot be dropped into the reacting area or absorbed into the reacting area easily.

Referring to FIG. 6 for the U.S. Pat. No. 5,997,817, a siphon type laminated electrode strip comprises an insulating substrate, an electrode system disposed on the same side, an insulating layer covered onto the electrode system and defining a reacting area and the anode and cathode connectors of a detector, a reacting membrane covered onto the reacting area of the electrodes, a hydrophilic thin membrane covered onto the top of the reacting membrane, and an upper cover plastic membrane having a transparent window of the size of the reacting area is attached. However, there is only one absorbing opening for the test sample of this strip and its direction is located on the right side, and thus having a shortcoming that the test sample can be applied to only one opening and its direction is fixed.

### SUMMARY OF THE INVENTION

In view of the shortcomings of the prior art, the inventor of the present invention aimed at the problem and tried to find a way of overcoming the shortcomings and conducted extensive researched and experiments to find a feasible solution, and finally invented the present invention.

Therefore, it is a primary object of the present invention to overcome the shortcomings of the prior art by providing a bioelectrochemical sensor strip capable of taking trace samples, and this strip comprises a reacting area at an end, and the reacting area is protruded from the strip and is fully open. Therefore, only a small quantity of test sample is needed to be absorbed into the reacting area by the capillary action.

To achieve the foregoing object, the present invention provides a bioelectrochemical sensor strip capable of taking trace samples comprising: a pair of insulating substrates, and the substrate is comprised of a first substrate and a second substrate; a reacting area protruded from an end of the first and second substrate, and the reacting area is transparent, and the periphery of the reacting area is fully open; a working electrode and a reference electrode disposed between the first and second substrates, and the electrodes are maintained with an appropriate distance apart, and an end of the electrode is extended into the reacting area, so that when the test is performed, the test sample can be absorbed into the reacting area easily by the capillary action without a limitation of directions, since the periphery of the reacting area is fully open. It is easy to take the blood sample, and the required quantity of the test sample is less than 3 μl, and thus can reduce the patient's pain of collecting the blood sample, and the quantity of the blood sample is constant which can lower the error percentage caused by the operation of different quantities of the test sample.

Another object of the present invention is to provide a bioelectrochemical sensor strip capable of taking trace samples that its reacting area includes a hydrophilic reacting membrane covered onto a working electrode and a reference electrode, and the reacting membrane is used for contacting the test sample to have a dedicated electrochemical reaction, and the reacting membrane is comprised of hydrophilic macromolecule and enzyme, so that the test sample can enter into the reacting area easily.

A further object of the invention is to provide a bioelectrochemical sensor strip capable of taking trace samples wherein the edge of the reacting area is a protruded structure, not only can be identified by its texture easily, but also can absorb the test sample easily to reduce dose hesitation occurred when the test sample enters into the reacting area.

### BRIEF DESCRIPTION OF THE DRAWINGS

To make it easier for our examiner to understand the objective, shape, assembly, structure, characteristics and performance of the present invention, the following embodiments accompanied with the related drawings are described in details.

FIG. 1 is a schematic view of U.S. Pat. No. 5,762,770;

FIG. 2 is a schematic view of U.S. Pat. No. 5,120,420;

FIG. 3 is a schematic view of U.S. Pat. No. 5,73,735;

FIG. 4 is a schematic view of U.S. Pat. No. 6,258,229B1;

FIG. 5 is a schematic view of R. 0. C. Pat. No. 570192;

FIG. 6 is a schematic view of U.S. Pat. No. 5,997,817

FIG. 7 is an exploded view of the present invention;

FIG. 8 is a perspective view of the present invention;

FIG. 9 is a planar view of the present invention; and

FIG. 10 is a cross-sectional view of the present invention.

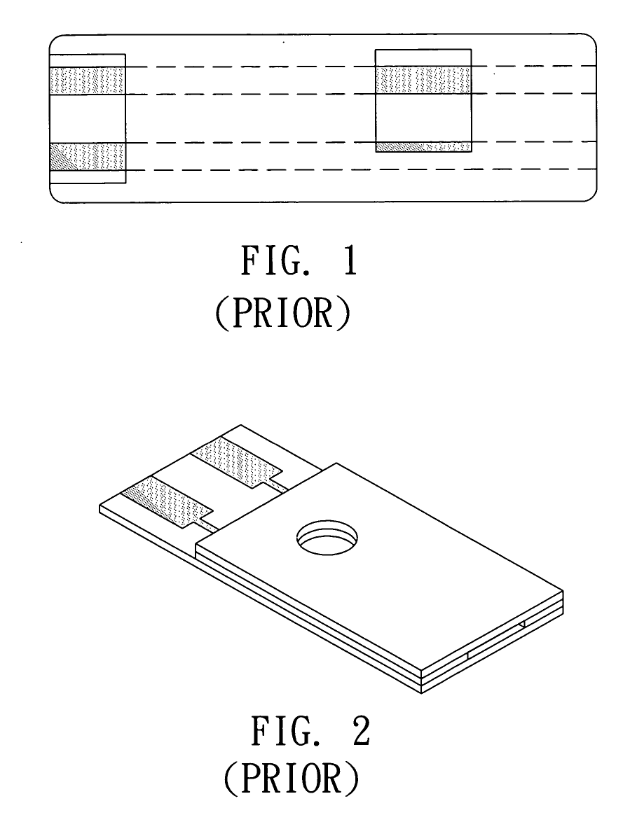

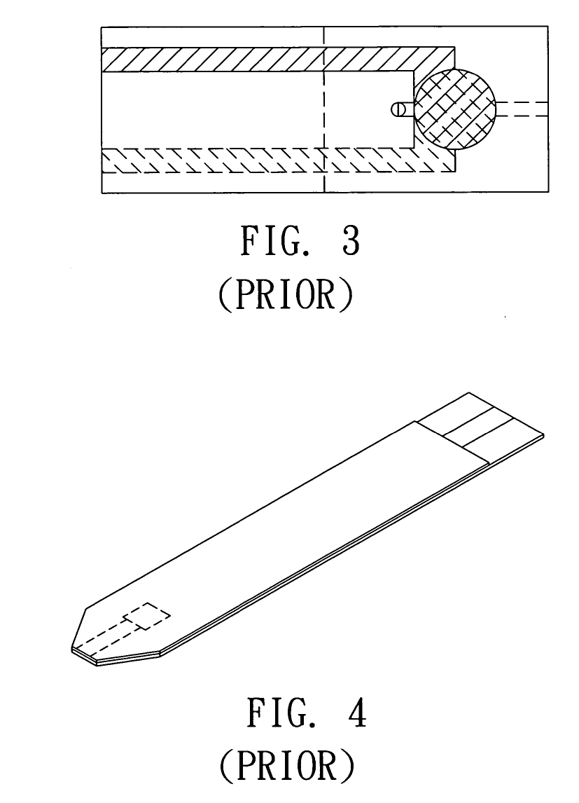

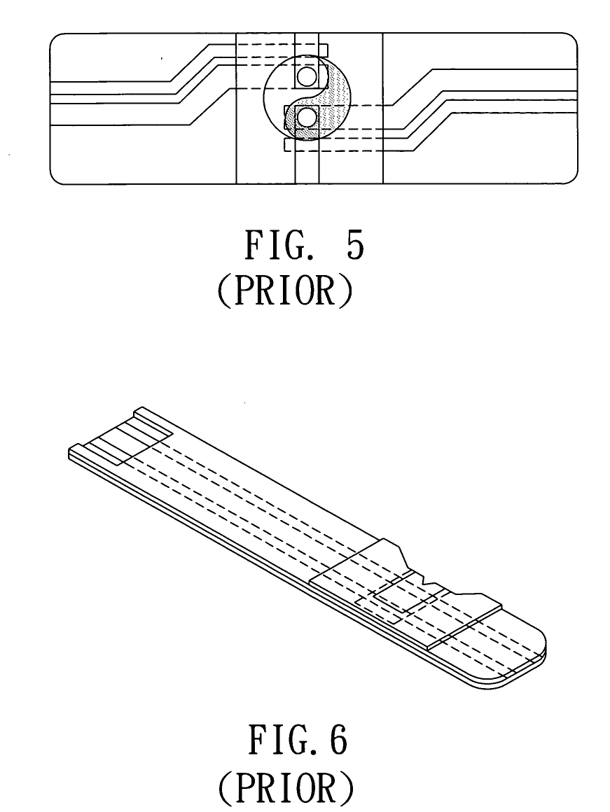

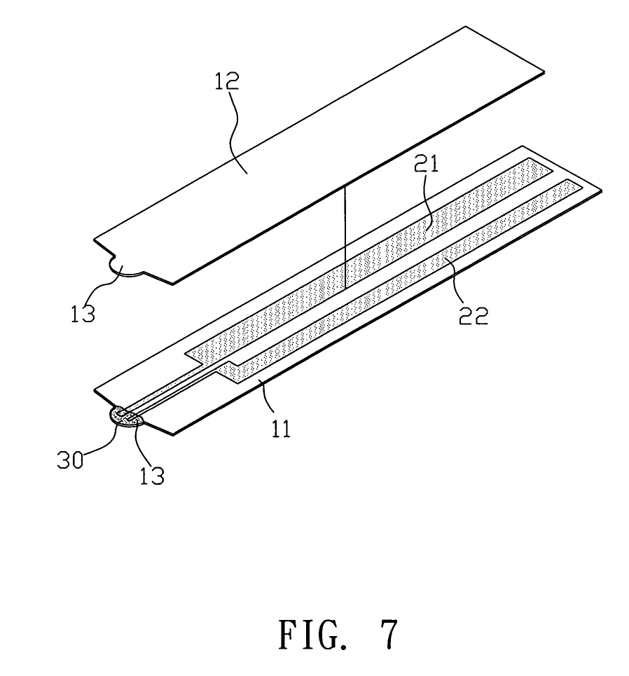

### DETAILED DESCRIPTION OF THE PREFERRED EMBODIMENTS

The present invention discloses a bioelectrochemical sensor strip capable of taking trace samples that uses an oxidizing matter or a reducing matter to have a dedicated bioelectrochemical reaction with the test sample to produce a bioelectrochemical sensor strip with an electron transfer. By the use of the testing instrument, the concentration of the test sample can be measured dedicatedly and sensitively.

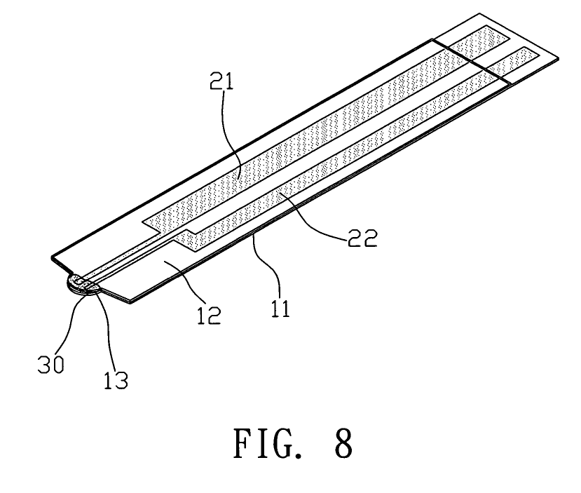

Referring to FIGS. 7 and 8, the strip includes a first and a second substrates 11, 12, the first and second substrates 11, 12 are in a laminated form and are electrically insulated. Besides both ends of the periphery, the first and second substrates 11, 12 include an adhesive glue on their corresponding opposite sides (however those skilled in the art can use other methods to substitute the adhesive glue) to glue the first and second substrates 11, 12 together. The portion without being covered by adhesive glue is hydrophilic, and one end of the second substrate 12 is not fully covered on the first substrate 11, and the other end of the first and second substrates 11, 12 includes an outwardly protruded reacting area 13. The reacting area 13 is transparent, and the periphery of the reacting area 13 is protruded, and the periphery of the reacting area 11 is fully open, not only allowing users to easily identify the test sample, but also being absorbed by the capillary action easily, so as to reduce the dose hesitation when the test sample enters into the reacting area as shown in FIG. 8.

Further, a working electrode 21 and a reference electrode 22 are disposed between the substrates 11, 12, and the working electrode 21 and reference electrode 22 are maintained with a specific distance apart, and an end of the working electrode 21 and the reference electrode 22 is extended deep into the reacting area 13, and the other end is protruded from the second substrate 12 which is shorter than the first substrate 11 and contained at the end of the first substrate 11.

Further, the hydrophilic reacting membrane 30 is covered onto the reacting area 13, and the working electrode 21 and the reference electrode 22 are covered onto the hydrophilic reacting membrane 30. In this preferred embodiment, the working electrode 21 and the reference electrode 22 are hydrophilic enzymes composed of an oxidizing matter or a reducing matter, a hydrophilic macromolecule, an electronic medium, an interface activator, and a buffering solution for contacting and having a biochemical reaction with the test sample.

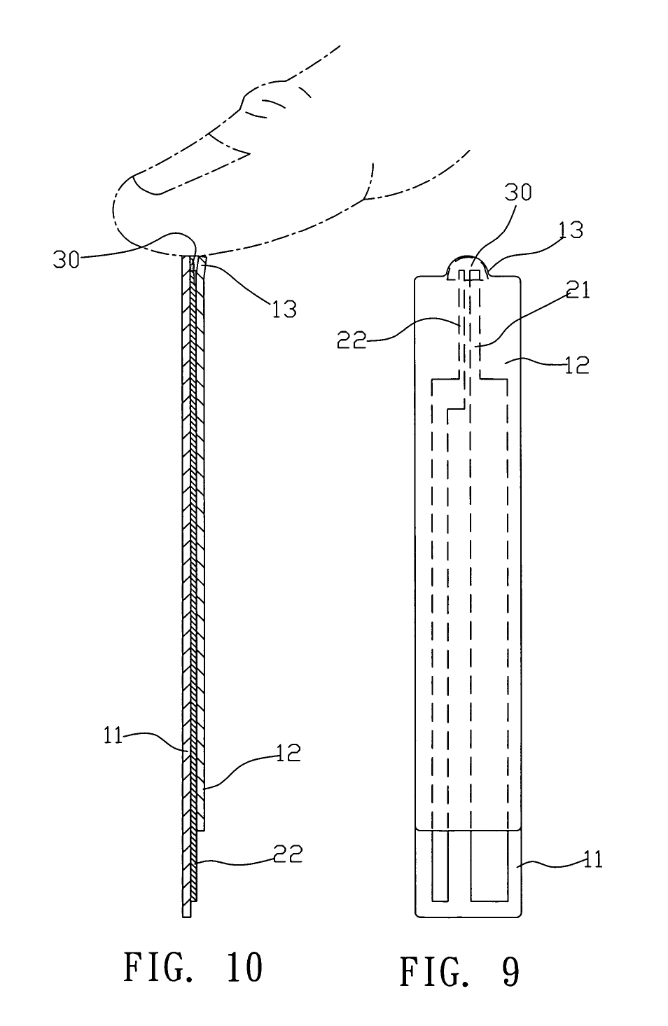

Referring to FIGS. 9 and 10 for the use of the sensor, the test sample can be absorbed into the reacting area 13 easily by the capillary action without being limited by the direction, since the reacting area 13 is protruded from the substrate and the periphery of the reacting area 13 is fully open. Furthermore, the test sample uses the hydrophilic reacting membrane 30 to introduce the test sample into the reacting area 13 for the biochemical reaction, and the required quantity of the sample is less than 3 μl and thus reducing the patient's pain of collecting the blood sample. The required quantity of the test sample is constant, and thus the invention can eliminate the measuring error caused by different quantities of the test sample each time for the operation.

In summation of the above description, the present invention herein enhances the performance and overcomes the shortcoming of the prior art, and further complies with the patent application requirements.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A bioelectrochemical sensor strip capable of taking trace samples, for testing the concentration of a biochemical matter in a test sample, comprising: a pair of insulating first and second substrates, attached with each other at the corresponding surface of their peripheries, and a working electrode and a reference electrode being disposed between said first and second substrates; and a reacting area, protruded from an end other than that where said first and second substrates are attached, and the periphery of said reacting area is open.

2. The bioelectrochemical sensor strip capable of taking trace samples of claim 1, wherein said reacting area comprises a hydrophilic reacting membrane covered on said working electrode and reference electrode for contacting and having a dedicated electrochemical reaction with said test sample, and said reacting membrane is comprised of said hydrophilic reacting membrane, such that said test sample is capable of entering said reacting area easily.

3. The bioelectrochemical sensor strip capable of taking trace samples of claim 2, wherein said hydrophilic reacting membrane is composed of an oxidizing enzyme or a reducing enzyme, a hydrophilic macromolecule, an electronic medium, an interface activator and a buffering liquid salt.
